(19) United States
(12) Patent Application Publication
(10) Pub. No.: US 2026/0069202 A1
(43) Pub. Date: Mar. 12, 2026

(54) Sleep Monitoring Using Non-Contact Sensors

(71) Applicant: Samsung Electronics Co., Ltd., Suwon-si (KR)

(72) Inventors: Hao-Hsuan Chang, Plano, TX (US); Hao Chen, Allen, TX (US); Se Ho Kim, Richardson, TX (US); Doyoon Kim, Suwon (KR)

(21) Appl. No.: 19/045,020

(22) Filed: Feb. 4, 2025

Related U.S. Application Data
(60) Provisional application No. 63/693,119, filed on Sep. 10, 2024.

(51) Int. Cl. A61B 5/00; A61B 5/05

(52) U.S. Cl. CPC A61B 5/4812; A61B 5/05; A61B 5/6887; A61B 5/7264; A61B 5/7275

(57) ABSTRACT

In one embodiment, a method includes obtaining a sensor signal from each of one or more non-contact sensors in an environment of a user and, for each sensor signal, extracting one or more physiological features of the user from that sensor signal. The method further includes for each sensor signal, embedding, by a trained encoder dedicated to the non-contact sensor corresponding to that sensor signal, the one or more physiological features extracted from that sensor signal into a joint sleep-stage embedding space; determining, based on a final embedding that is based at least in part on the embedded physiological features, a similarity between the final embedding and each of multiple sleep-stage embeddings, each identifying a predetermined sleep stage of a person; and predicting, based on the similarities between the final embedding and the sleep-stage embeddings, a sleep stage of the user.

### PRIORITY CLAIM

This application claims the benefit under 35 U.S.C. § 119 of U.S. Provisional Patent Application No. 63/693,119 filed Sep. 10, 2024, which is incorporated by reference herein.

### TECHNICAL FIELD

This application generally relates to sleep monitoring using one or more non-contact sensors.

### BACKGROUND

In humans, sleep can be divided into several distinct stages. For instance, the following sleep stages may be used to define sleep in humans with the aid of polysomnographic (PSG) techniques: (1) a wakeful, non-sleep stage; (2) an initial light-sleep stage, also referred to as an “N1” stage; (3) a subsequent, deeper-sleep stage, also referred to as an “N2” stage; (4) a slow-wave sleep stage, also referred to as a “slow-wave” sleep stage; and (5) a rapid-eye movement, or “REM,” sleep stage, although there are other ways of defining human sleep stages. A typical night of sleep in an adult human can include 4-6 rounds of sleep cycles, with each sleep cycle including the four stages 2-5 described above.

Sleep is an important factor in human health and wellbeing, and detecting a person's sleep stage can play an important role in understanding that person's sleep patterns and diagnosing any sleep-related conditions in the person. PSG involves monitoring a person's sleep patterns through the use of contact sensors on various parts of the person's body, and these contacts sensors are used to detect brain waves, electromyography, body movement, and so on. A trained healthcare professional reviews the PSG data over time to determine which sleep stage a person is at any given time, and how much time a person spends in various sleep stages.

### BRIEF DESCRIPTION OF THE DRAWINGS

FIG. 1 illustrates an example method for predicting a sleep stage of a person using one or more non-contact sensors.

FIG. 2 illustrates an example of certain steps of the example method of FIG. 1 for N non-contact sensors, as well as a training process for training encoders.

FIG. 3 illustrates an example AI fusion model for generating a final embedding, as well as an approach for training an AI fusion model.

FIG. 4 illustrates an example in which a fusion embedding generated during an inference stage is compared using a similarity function to sleep-stage embeddings.

FIG. 5 illustrates an example of a domain adaptation algorithm to transfer the knowledge from an accessed sleep dataset to non-contact sensor-based sleep-stage prediction.

FIG. 6 illustrates an example computing system.

### DESCRIPTION OF EXAMPLE EMBODIMENTS

Sleep-stage classification is an important process for the evaluation of sleep quality in a clinical sleep study. One typical night of sleep for an adult can include four to six rounds of a sleep cycle, which is made up of different sleep stages; including, for example, wake, light sleep, deep sleep, and rapid-eye movement (REM) sleep. A typical clinical sleep study evaluates the sleep quality of a patient based on whole-night polysomnographic (PSG) recordings, which includes contact sensors for electroencephalogram (EEG), electromyogram (EMG), electrooculogram (EOG), pulse oximetry, and airflow, etc. After PSG recordings, sleep specialists assess the sleep quality by scoring sleep stages.

The typical sleep study causes discomfort for patients because many electrodes and devices are attached to patients. Furthermore, it is also labor-intensive and time-consuming as these medical devices have to be operated by trained technicians and the sleep stages are manually labeled by medical experts. In addition, sleep studies occur in a clinical environment, and therefore sleep evaluation is not feasible when the patient is in other, more typical settings (e.g., when the patient is in the patient's residence bedroom).

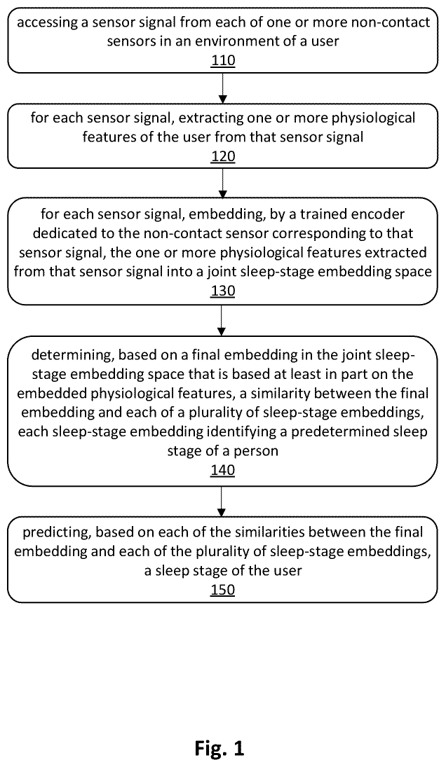

In contrast, this disclosure describes techniques for determining sleep stages of a person using one or more non-contact sensors. FIG. 1 illustrates an example method for predicting a sleep stage of a person using one or more non-contact sensors. Types of non-contact sensors for non-invasively predicting a sleep stage of a user include a radar (e.g., millimeter wave), a microphone, a sonar, a thermometer, a humidity sensor, a pressure sensor, etc. For instance, a pressure sensor such as a piezoelectric sensor may be placed in or under a sleeping surface of a user (e.g., in or under a mattress) and may detect changes in pressure, which are correlated with movement of the user. As another example, a radar/radio sensor may transmit a low-power radio signal and record the reflections from a user, and these reflections correspond to vital signs such as breathing rates or heart beats, and such cardio-respiratory signals are highly correlated with sleep stages. As another example, a microphone may record sounds from a user, and a thermometer (e.g., an infrared thermometer) may record temperature in an environment of user (e.g., a temperature of the user's sleeping room, or the temperature of the user).

In particular embodiments, non-contact sensors may be located in an electronic device in the environment of the user. For example, one or more non-contact sensors (e.g., a radar, such as millimeter-wave radar) may be located in an HVAC component, such as a heater or an air conditioner. As another example, one or more wireless sensors may be located in a smart TV, a smartphone, a radio, etc. In particular embodiments, one or more non-contact sensors may be embedded in non-electronic devices, such as in furniture (e.g., a nightstand, a bed, a chair, etc.) in the user's environment.

Step 110 of the example method of FIG. 1 includes accessing a sensor signal from each of one or more non-contact sensors in an environment of a user. The sensor signal obtained from a non-contact sensor may be obtained over a period of time or over several periods of time (e.g., the sensor may cycle on and off, collecting signals during each “on” time). Step 120 of the example method of FIG. 1 includes, for each sensor signal, extracting one or more physiological features of the user from that sensor signal. In other words, one or more physiological features are extracted from each sensor signal obtained from a particular non-non-contact sensor. For instance, step 120 may include extracting a user's body movements from pressure readings over time obtained by a pressure sensor, e.g., placed under a user's mattress. As another example step 120 may include extracting a user's cardio-respiratory information from reflected radar signals.

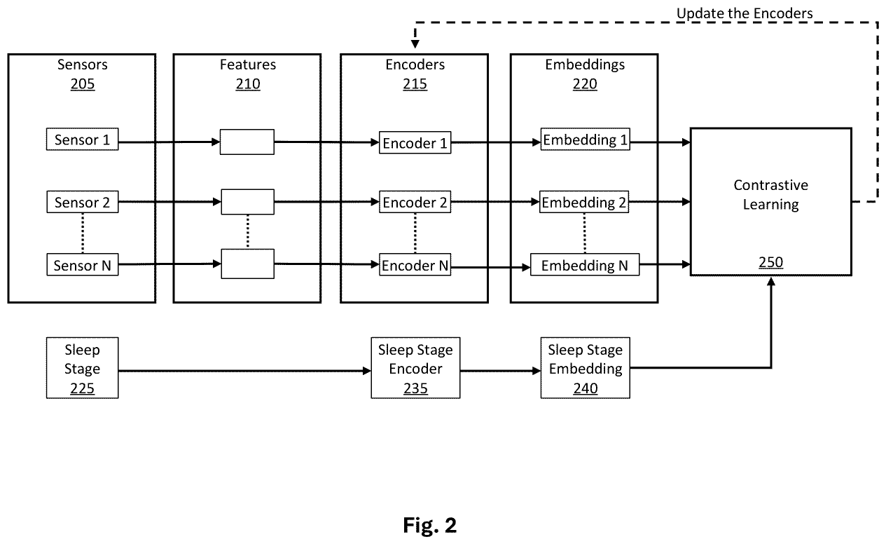

Step 130 of the method of FIG. 1 includes for, each sensor signal, embedding, by a trained encoder dedicated to the non-contact sensor corresponding to that sensor signal, the one or more physiological features extracted from that sensor signal into a joint sleep-stage embedding space. FIG. 2 illustrates an example of steps 110-130 for N non-contact sensors, as well as a training process for training the encoders, as described more fully below. In the example of FIG. 2, non-contact sensors 205 are in the environment of a user and acquire sensor signals. These sensor signals are used to extract physiological features 210. As illustrated in FIG. 2 and described in FIG. 1, the output of each non-contact sensor is used to extract a set of one or more physiological features, i.e., a set of one or more physiological features is extracted from each sensor signal. Each set of physiological features is input to one of encoders 215. As illustrated in FIG. 2, each encoder is dedicated to a particular non-contact sensor, e.g., in FIG. 2, encoder 1 is dedicated to sensor 1, encoder 2 is dedicated to sensor 2, and encoder N is dedicated to sensor N, so that each encoder encodes the set of physiological signals corresponding to its non-contact sensor. Encoders 215 generate embeddings 220, and as illustrated in FIG. 2, each encoder generates a particular embedding (e.g., encoder 1 generates embedding 1, and so on). Thus, each encoder generates an embedding of the physiological signals extracted from the sensor signals of the non-contact sensor that the encoder is dedicated to.

Each encoder embeds its input physiological signals in a joint sleep-stage embedding space. In other words, the embedding space is shared by (is the same for) all of the encoders, as well as for the sleep-stage encoder, which is described more fully below. As a result, different non-contact sensors may correspond to different physiological signals, but these signals would still be embedded into the same space.

FIG. 2 illustrates an example training procedure for encoders 215. First, a set of training data is obtained, where the training data includes (1) sensor output for a user from each of the sensors corresponding to the encoders to be trained and (2) ground-truth sleep stage labels for the periods of time coincident with the sensor output. The ground-truth labels may be obtained by, for example, PSG data as determined by a medical professional. While PSG data is being obtained for a particular patient, then training-data sensor signals from one or more non-contact sensors may simultaneously be obtained.

For each non-contact sensor from which training data was collected, then a set of one or more physiological signals are extracted from the training-data sensor signals. These physiological signals are input to their corresponding untrained encoders. Likewise, as illustrated in FIG. 2, the sleep-stage labels 225 for the joint sleep-stage embedding space are input to a sleep stage encoder 235. The sleep-stage encoder 235 (which may be multiple encoders, in particular embodiments) generates sleep-stage embeddings from the sleep-stage labels. Sleep-stage embeddings are embedded in the joint sleep-stage embedding space; for example, each sleep-stage embedding 240 may be a centroid of a cluster in the embedding space, where each cluster corresponds to the data collected for a particular sleep-stage label.

Once the training data embeddings and the sleep-stage embeddings are obtained, then the untrained encoders may be trained using constrastive learning 250. For example, the embeddings of each encoder and the sleep-stage embeddings are input to contrastive learning block 250 to determine a constrastive loss. For instance, a constrastive loss for a particular sensor/encoder embedding may be:

$${L}_{InfoNCE} = - {E}_{X} [ log \frac{{f}_{S i m} ( {s}^{+} , x )}{{\sum }_{j = 1}^{N - 1} {f}_{S i m} ( {s}_{j}^{-} , x )} ] \qquad ( 1 )$$

where x is the sensor embedding, s⁺ is the sleep embedding of the ground-truth sleep label,

$${s}_{j}^{-}$$

is all sleep embeddings except for s⁺, and fₛᵢₘ(x, y) is the similarity function of two embeddings x, y. In particular embodiments, the similarity function may be the cosine similarity between two embeddings. In other embodiments the similarity function may be the negative Euclidean distance between two embeddings, and this disclosure contemplates that any suitable similarity metric may be used. Each encoder is updated to output the embeddings that minimize the contrastive loss. As a result, while each encoder embeds signals from its sensor into a joint embedding space, contrastive learning block 250 tends to aligns embeddings with their ground-truth sleep stages and separate embeddings from the other sleep embeddings in the joint embedding space, e.g., so that data representing different sleep stages corresponds to different clusters in the joint embedding space. Once trained, the encoders can then be employed for inference to accurately predict a particular person's sleep stages in a non-invasive manner.

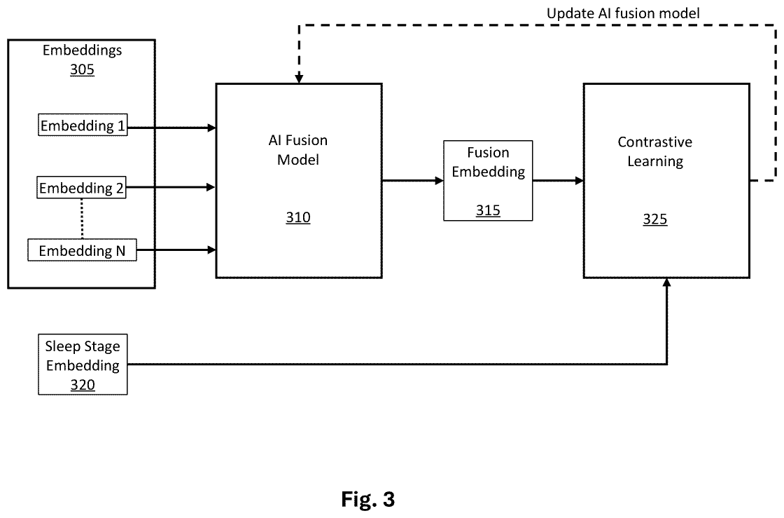

Step 140 of the method of FIG. 1 includes determining, based on a final embedding in the joint sleep-stage embedding space that is based at least in part on the embedded physiological features, a similarity between the final embedding and each of a plurality of sleep-stage embeddings, each sleep-stage embedding identifying a predetermined sleep stage of a person. In particular embodiments that use a single non-contact sensor (e.g., because only one such non-contact sensor is present, or is powered on, or is providing sufficiently high-quality signals), then the final embedding is the embedded physiological signals from the encoder for that sensor. In multimodal embodiments that predict a user's sleep stage at a given time based on signals from more than one non-contact sensor, then the final embedding is based on each of the embeddings from the encoders corresponding to each of those sensors. For instance, FIG. 3 illustrates an example AI fusion model 310 for generating a final embedding. To generate a final embedding, a trained AI fusion model 310 takes each embedding from the corresponding encoders and then creates a final embedding, for example by learning a mapping function in the joint embedding space. For instance, the embeddings from each encoder may be concatenated, and this concatenated vector may be provided as input to the trained AI fusion model (e.g., a trained feedforward neural network) to output the final, fused embedding (e.g., fusion embedding 315) in the joint sleep-stage embedding space.

FIG. 3 specifically illustrates an approach for training AI fusion model 310. Embeddings 305 from the encoders corresponding to non-contact sensors are input to the AI fusion model, which generates a final fusion embedding 315. Sleep-stage embeddings and the fusion embedding 315 are provided to contrastive learning block 325, which updates the parameters (e.g., model weights) of AI fusion model 310 based on a loss determined from the input fusion embedding and sleep-stage embeddings, for example using the loss function of Eq. 1, above, so that the AI fusion model 310 learns to output a fusion embedding that aligns with the corresponding ground-truth sleep stage for a particular training sample.

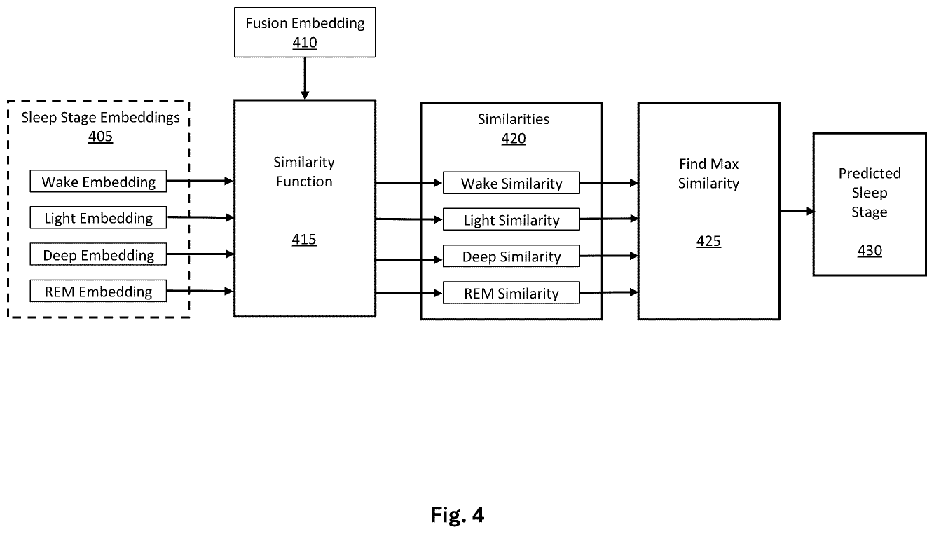

Once trained, the trained AI fusion model can be deployed for inferencing when multiple non-contact sensors are used to predict a user's sleep stage. FIG. 4 illustrates an example in which a fusion embedding 410 generated during an inference stage is compared using a similarity function 415 to each of the sleep-stage embeddings 405 for an example predetermined set of sleep stages. As illustrated in FIG. 4, a set of similarities 420 is then generated, one similarity for each sleep-stage embedding (i.e., a separate similarity measure between the fusion embedding 410 and each sleep-stage embedding). Step 150 of the example method of FIG. 1 includes predicting, based on each of the similarities between the final embedding and each of the plurality of sleep-stage embeddings, a sleep stage of the user, and in the example of FIG. 4, the predicted sleep stage 430 is determined by finding the maximum (greatest) similarity 425. Similarity function 415 is f_{Sim}(x, y), i.e., the same similarity function as used during model learning.

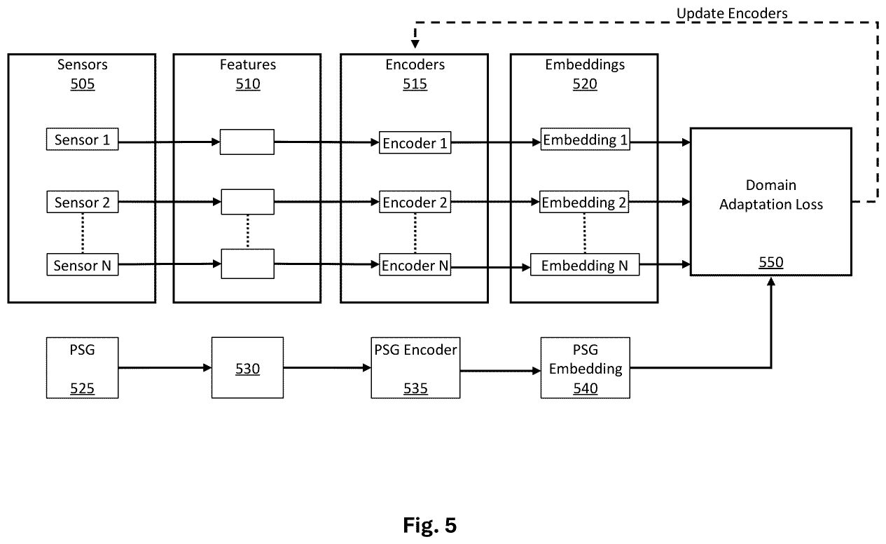

Collecting sleep-stage training data can be a labor-intensive and time-consuming process. Therefore, in particular embodiments, collected sensor training data with ground-truth sleep stages may be limited and may be insufficient for training an AI fusion model for sleep stage prediction. Therefore, particular embodiments may utilize an existing sleep dataset, such as a public sleep dataset with sleep stage labels and polysomnographic (PSG) recordings, to facilitate AI model training. FIG. 5 illustrates an example of a domain adaptation algorithm to transfer the knowledge from this accessed sleep dataset to non-contact sensor-based sleep-stage prediction. As illustrated in FIG. 5, sensor signals from sensors 505 are collected and features 510 are extracted, as described above. These features are encoded by encoders 515, which output embeddings 520. PSG data 525 from the dataset is accessed and physiological features 530 are determined from the PSG data. These features are then embedded by PSG encoder 535 into the joint sleep-stage embedding space to produce PSG embedding 540. Embeddings 520 and 540 are input to domain adaptation loss block 550 to compute a domain adaptation loss, and the sensor encoders are then updated to minimize this domain adaptation loss. In one example of the domain adaptation loss can be supervised domain adaptation (SDA) loss:

$${ℒ}_{S D A} = {\sum }_{e}^{E} {x}_{l a b e l} * {x}_{dist}^{2} + ( 1 - {x}_{l a b e l} ) * {max ( l - {x}_{dist} , 0 )}^{2} \qquad ( 2 )$$

where x_{label}=1 if the sensor embedding and the PSG embedding have the same sleep stage, otherwise x_{label}=0, x_{dist} is the distance between sensor embedding (xₛₑₙₛₒᵣ) and PSG embedding (x_{PSG}). Once example of a distance function is the Euclidean distance, which is x_{dist}=∥xₛₑₙₛₒᵣ−X_{PSG}∥², although this disclosure contemplates that other distance functions may be used. The SDA loss is to align the PSG embedding and sensor embedding with the same sleep stage while separating the PSG embedding and sensor embedding that have different sleep stages. Finally, the sensor encoders are updated to output the embeddings that minimize the SDA loss.

The step of example FIG. 1 may be performed by one device or by more than one device. For example, a consumer or client device (e.g., an air conditioner or a smart TV, etc.) that contains at least one non-contact sensor may also perform all or some of the steps of FIG. 1. In particular embodiments, device(s) that contain non-contact sensors may transmit their sensor signals (or processed variants thereof, e.g., extracted features) to another client device (e.g., a smartphone) or server device to preform some or all of the steps of FIG. 1. In particular embodiments, one or more client-side devices may collectively perform some of the steps of the method of FIG. 1, while one or more server-side devices perform other steps of the method of FIG. 1. In a cloud-based embodiment, once sensor data is collected by the non-contact sensors, then one or more server devices may perform all the steps of FIG. 1.

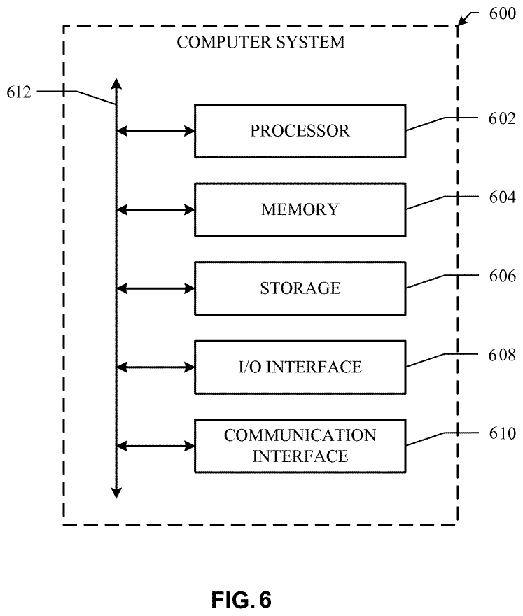

FIG. 6 illustrates an example computer system 600. In particular embodiments, one or more computer systems 600 perform one or more steps of one or more methods described or illustrated herein. In particular embodiments, one or more computer systems 600 provide functionality described or illustrated herein. In particular embodiments, software running on one or more computer systems 600 performs one or more steps of one or more methods described or illustrated herein or provides functionality described or illustrated herein. Particular embodiments include one or more portions of one or more computer systems 600. Herein, reference to a computer system may encompass a computing device, and vice versa, where appropriate. Moreover, reference to a computer system may encompass one or more computer systems, where appropriate.

This disclosure contemplates any suitable number of computer systems 600. This disclosure contemplates computer system 600 taking any suitable physical form. As example and not by way of limitation, computer system 600 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 600 may include one or more computer systems 600; be unitary or distributed; span multiple locations; span multiple machines; span multiple data centers; or reside in a cloud, which may include one or more cloud components in one or more networks. Where appropriate, one or more computer systems 600 may perform without substantial spatial or temporal limitation one or more steps of one or more methods described or illustrated herein. As an example and not by way of limitation, one or more computer systems 600 may perform in real time or in batch mode one or more steps of one or more methods described or illustrated herein. One or more computer systems 600 may perform at different times or at different locations one or more steps of one or more methods described or illustrated herein, where appropriate.

In particular embodiments, computer system 600 includes a processor 602, memory 604, storage 606, an input/output (I/O) interface 608, a communication interface 610, and a bus 612. Although this disclosure describes and illustrates a particular computer system having a particular number of particular components in a particular arrangement, this disclosure contemplates any suitable computer system having any suitable number of any suitable components in any suitable arrangement.

In particular embodiments, processor 602 includes hardware for executing instructions, such as those making up a computer program. As an example and not by way of limitation, to execute instructions, processor 602 may retrieve (or fetch) the instructions from an internal register, an internal cache, memory 604, or storage 606; decode and execute them; and then write one or more results to an internal register, an internal cache, memory 604, or storage 606. In particular embodiments, processor 602 may include one or more internal caches for data, instructions, or addresses. This disclosure contemplates processor 602 including any suitable number of any suitable internal caches, where appropriate. As an example and not by way of limitation, processor 602 may include one or more instruction caches, one or more data caches, and one or more translation lookaside buffers (TLBs). Instructions in the instruction caches may be copies of instructions in memory 604 or storage 606, and the instruction caches may speed up retrieval of those instructions by processor 602. Data in the data caches may be copies of data in memory 604 or storage 606 for instructions executing at processor 602 to operate on; the results of previous instructions executed at processor 602 for access by subsequent instructions executing at processor 602 or for writing to memory 604 or storage 606; or other suitable data. The data caches may speed up read or write operations by processor 602. The TLBs may speed up virtual-address translation for processor 602. In particular embodiments, processor 602 may include one or more internal registers for data, instructions, or addresses. This disclosure contemplates processor 602 including any suitable number of any suitable internal registers, where appropriate. Where appropriate, processor 602 may include one or more arithmetic logic units (ALUs); be a multi-core processor; or include one or more processors 602. Although this disclosure describes and illustrates a particular processor, this disclosure contemplates any suitable processor.

In particular embodiments, memory 604 includes main memory for storing instructions for processor 602 to execute or data for processor 602 to operate on. As an example and not by way of limitation, computer system 600 may load instructions from storage 606 or another source (such as, for example, another computer system 600) to memory 604. Processor 602 may then load the instructions from memory 604 to an internal register or internal cache. To execute the instructions, processor 602 may retrieve the instructions from the internal register or internal cache and decode them. During or after execution of the instructions, processor 602 may write one or more results (which may be intermediate or final results) to the internal register or internal cache. Processor 602 may then write one or more of those results to memory 604. In particular embodiments, processor 602 executes only instructions in one or more internal registers or internal caches or in memory 604 (as opposed to storage 606 or elsewhere) and operates only on data in one or more internal registers or internal caches or in memory 604 (as opposed to storage 606 or elsewhere). One or more memory buses (which may each include an address bus and a data bus) may couple processor 602 to memory 604. Bus 612 may include one or more memory buses, as described below. In particular embodiments, one or more memory management units (MMUs) reside between processor 602 and memory 604 and facilitate accesses to memory 604 requested by processor 602. In particular embodiments, memory 604 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 604 may include one or more memories 604, where appropriate. Although this disclosure describes and illustrates particular memory, this disclosure contemplates any suitable memory.

In particular embodiments, storage 606 includes mass storage for data or instructions. As an example and not by way of limitation, storage 606 may include a hard disk drive (HDD), a floppy disk drive, flash memory, an optical disc, a magneto-optical disc, magnetic tape, or a Universal Serial Bus (USB) drive or a combination of two or more of these. Storage 606 may include removable or non-removable (or fixed) media, where appropriate. Storage 606 may be internal or external to computer system 600, where appropriate. In particular embodiments, storage 606 is non-volatile, solid-state memory. In particular embodiments, storage 606 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 606 taking any suitable physical form. Storage 606 may include one or more storage control units facilitating communication between processor 602 and storage 606, where appropriate. Where appropriate, storage 606 may include one or more storages 606. Although this disclosure describes and illustrates particular storage, this disclosure contemplates any suitable storage.

In particular embodiments, I/O interface 608 includes hardware, software, or both, providing one or more interfaces for communication between computer system 600 and one or more I/O devices. Computer system 600 may include one or more of these I/O devices, where appropriate. One or more of these I/O devices may enable communication between a person and computer system 600. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 608 for them. Where appropriate, I/O interface 608 may include one or more device or software drivers enabling processor 602 to drive one or more of these I/O devices. I/O interface 608 may include one or more I/O interfaces 608, where appropriate. Although this disclosure describes and illustrates a particular I/O interface, this disclosure contemplates any suitable I/O interface.

In particular embodiments, communication interface 610 includes hardware, software, or both providing one or more interfaces for communication (such as, for example, packet-based communication) between computer system 600 and one or more other computer systems 600 or one or more networks. As an example and not by way of limitation, communication interface 610 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 610 for it. As an example and not by way of limitation, computer system 600 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 600 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 600 may include any suitable communication interface 610 for any of these networks, where appropriate. Communication interface 610 may include one or more communication interfaces 610, where appropriate. Although this disclosure describes and illustrates a particular communication interface, this disclosure contemplates any suitable communication interface.

In particular embodiments, bus 612 includes hardware, software, or both coupling components of computer system 600 to each other. As an example and not by way of limitation, bus 612 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 612 may include one or more buses 612, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
accessing a sensor signal from each of one or more non-contact sensors in an environment of a user;
for each sensor signal, extracting one or more physiological features of the user from that sensor signal; and
for each sensor signal, embedding, by a trained encoder dedicated to the non-contact sensor corresponding to that sensor signal, the one or more physiological features extracted from that sensor signal into a joint sleep-stage embedding space;
determining, based on a final embedding in the joint sleep-stage embedding space that is based at least in part on the embedded physiological features, a similarity between the final embedding and each of a plurality of sleep-stage embeddings, each sleep-stage embedding identifying a predetermined sleep stage of a person; and
predicting, based on each of the similarities between the final embedding and each of the plurality of sleep-stage embeddings, a sleep stage of the user.

2. The method of claim 1, wherein at least one of the one or more non-contact sensors is part of (1) an air conditioner or (2) a smart TV.

3. The method of claim 2, wherein the at least one non-contact sensor comprises a millimeter-wave radar sensor.

4. The method of claim 1, wherein:
the one or more non-contact sensors comprise a plurality of non-contact sensors; and
the method further comprises:
inputting, to a trained AI fusion model, each of the embeddings; and
generating, by the trained AI fusion model and based on each of the embeddings, the final embedding.

5. The method of claim 4, wherein the trained AI fusion model is trained based on a contrastive loss between (1) a plurality of training final embeddings output by the AI fusion model and (2) the sleep-stage embeddings.

6. The method of claim 1, wherein the joint sleep-stage embedding space contains a plurality of clusters, each cluster corresponding to one of the predetermined sleep stages.

7. The method of claim 6, wherein each trained encoder is trained by:
collecting, for the non-contact sensor corresponding to that encoder, a plurality of training data and corresponding ground-truth sleep-stage labels;
embedding, by the encoder, the training data in the embedding space; and
updating the encoder based on a contrastive loss that aligns the encoder embeddings with the embedded corresponding ground-truth sleep-stage labels.

8. The method of claim 1, further comprising:
repeating the steps of claim 1 over a period of time to generate a plurality of predicted sleep stages of the user for the period of time; and
determining, based the plurality of predicted sleep stages of the user, a sleep quality of the user for the period of time.

9. The method of claim 1, wherein the one or more non-contact sensors comprise one or more of a radar, a microphone, a sonar, a thermometer, a humidity sensor, or a pressure sensor.

10. A system comprising:
one or more non-contact sensors in an environment of a user; and
one or more non-transitory computer readable storage media storing instructions; and one or more processors coupled to the one or more non-transitory computer readable storage media and operable to execute the instructions to:
access a sensor signal from each of the one or more non-contact sensors;
for each sensor signal, extract one or more physiological features of the user from that sensor signal; and
for each sensor signal, embed, by a trained encoder dedicated to the non-contact sensor corresponding to that sensor signal, the one or more physiological features extracted from that sensor signal into a joint sleep-stage embedding space;
determine, based on a final embedding in the joint sleep-stage embedding space that is based at least in part on the embedded physiological features, a similarity between the final embedding and each of a plurality of sleep-stage embeddings, each sleep-stage embedding identifying a predetermined sleep stage of a person; and
predict, based on each of the similarities between the final embedding and each of the plurality of sleep-stage embeddings, a sleep stage of the user.

11. The system of claim 10, wherein at least one of the one or more non-contact sensors is part of (1) an air conditioner or (2) a smart TV.

12. The system of claim 11, wherein the at least one non-contact sensor comprises a millimeter-wave radar sensor.

13. The system of claim 10, wherein:
the one or more non-contact sensors comprise a plurality of non-contact sensors; and
further comprising one or more processors that are operable to execute the instructions to:
input, to a trained AI fusion model, each of the embeddings; and
generate, by the trained AI fusion model and based on each of the embeddings, the final embedding.

14. The system of claim 13, wherein the trained AI fusion model is trained based on a contrastive loss between (1) a plurality of training final embeddings output by the AI fusion model and (2) the sleep-stage embeddings.

15. The system of claim 10, wherein the joint sleep-stage embedding space contains a plurality of clusters, each cluster corresponding to one of the predetermined sleep stages.

16. The system of claim 15, wherein each trained encoder is trained by:
collecting, for the non-contact sensor corresponding to that encoder, a plurality of training data and corresponding ground-truth sleep-stage labels;
embedding, by the encoder, the training data in the embedding space; and
updating the encoder based on a contrastive loss that aligns the encoder embeddings with the embedded corresponding ground-truth sleep-stage labels.

17. The system of claim 10, further comprising one or more processors that are operable to execute the instructions to:
repeat the operations of claim 10 over a period of time to generate a plurality of predicted sleep stages of the user for the period of time; and
determine, based the plurality of predicted sleep stages of the user, a sleep quality of the user for the period of time.

18. The system of claim 10, wherein the one or more non-contact sensors comprise one or more of a radar, a microphone, a sonar, a thermometer, a humidity sensor, or a pressure sensor.

19. One or more non-transitory computer readable storage media storing instructions that are operable when executed to:
access a sensor signal from each of one or more non-contact sensors in an environment of a user;
for each sensor signal, extract one or more physiological features of the user from that sensor signal; and
for each sensor signal, embed, by a trained encoder dedicated to the non-contact sensor corresponding to that sensor signal, the one or more physiological features extracted from that sensor signal into a joint sleep-stage embedding space;
determine, based on a final embedding in the joint sleep-stage embedding space that is based at least in part on the embedded physiological features, a similarity between the final embedding and each of a plurality of sleep-stage embeddings, each sleep-stage embedding identifying a predetermined sleep stage of a person; and
predict, based on each of the similarities between the final embedding and each of the plurality of sleep-stage embeddings, a sleep stage of the user.

20. The media of claim 19, wherein at least one of the one or more non-contact sensors is part of (1) an air conditioner or (2) a smart TV.
